SEQUENCE LISTING (10 sequences)

SEQ ID NO: 1 - DNA, 560 bp, Homo sapiens
tgagaacaggactgagtgatggcttccaactccagcgatgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgc

SEQ ID NO: 2 - protein, 181 aa, Homo sapiens
MASNSSDVKEFLAKAKEDFLKKWESPAQNTAHLDQFERIKTLGTGSFGRVMLVKHKETGNHYAMKILDKQKVVKLKQIEHTLNEKRILQAVNFPFLVKLEFSFKDNSNLYMVMEYVPGGEMFSHLRRIGRFSEPHARFYAAQIVLTFEYLHSLDLIYRDLKPENLLIDQQGYIQVTDFGFA

SEQ ID NO: 3 - protein, 7 aa, Mus sp.
MPSSSND

SEQ ID NO: 4 - DNA, 27 bp, Artificial Sequence
  Description of Artificial Sequence Primer
ggacttggcctctcctgttcccttttg

SEQ ID NO: 5 - DNA, 25 bp, Artificial Sequence
  Description of Artificial Sequence Primer
tgtggggaaagaggaagggaaaagt

SEQ ID NO: 6 - DNA, 25 bp, Artificial Sequence
  Description of Artificial Sequence Primer
aaactgatccaagtgggctgtgttc

SEQ ID NO: 7 - DNA, 23 bp, Artificial Sequence
  Description of Artificial Sequence Primer
gcgaaaccgaagtctgtcacctg

SEQ ID NO: 8 - DNA, 40 bp, Artificial Sequence
  Description of Artificial Sequence Probe
aagaagggcagcgagcaggagagcgtgaaagaattcttag

SEQ ID NO: 9 - DNA, 40 bp, Artificial Sequence
  Description of Artificial Sequence Probe
ctgagaacaggactgagtgatggcttccaactccagcgat

SEQ ID NO: 10 - protein, 9 aa, Artificial Sequence
  Description of Artificial Sequence Synthetic peptide
ASNSSDVKE

(19) United States
(12) Patent Application Publication
(10) Pub. No.: US 2004/0106181 A1
(43) Pub. Date: Jun. 3, 2004

(54) Novel isoform of human camp-dependent protein kinase, calpha -s , localizes to sperm mid-piece and the use thereof

(76) Inventors: Nils Reinton, Oslo (NO); Sigurd Orstavik, Oslo (NO); Trine B Haugen, Oslo (NO); Tore Jahnsen, Ski (NO); Kjelil Tasken, Rykkin (NO); Bjorn S Skalhegg, Engerjordet (NO)

Correspondence Address: YOUNG & THOMPSON, 745 SOUTH 23RD STREET 2ND FLOOR, ARLINGTON, VA, 22202

(21) Appl. No.: 10/168,624

(22) PCT Filed: Dec. 21, 2000
(86) PCT No.: PCT/NO00/00441

(30) Foreign Application Priority Data
Dec. 23, 1999 (NO) 19996424

(51) Int. Cl. C12N 9/12; C07H 21/04

(52) U.S. Cl. 435/194; 435/69.1; 435/320.1; 435/325; 536/23.2

(57) ABSTRACT

The cloning of a novel human Cα isoform, identified as the human Cα-s protein on the basis of its similarity to a recently characterized Cα isoform which was identified by purification and peptide sequencing of Cα from ovine sperm. The human Cα-s protein was present in testis and ejaculated sperm and Cα-s localized to the midpiece of human sperm. A cDNA sequence encodes a Cα-s protein and includes the specific nucleotide sequence shown in SEQ ID NO:1, wherein the protein is a new splice variant of Cα catalytic subunit of cAMP dependent protein kinase.

### FIELD OF THE INVENTION

The present invention relates to a complementary DNA sequence encoding the Cα-s protein and a vector comprising said DNA sequence. This invention is also directed to said protein and the use of this protein in diagnosis, treatment and in developing male contraceptives.

### BACKGROUND OF THE INVENTION

Activation of cAMP-dependent protein kinase (PKA) by cAMP elicits initiation and maintenance of flagellar movement in mature spermatozoa [1;2]. The PKA tetrameric holoenzyme, composed of two catalytic subunits (C) and a regulatory subunit (R) dimer is activated upon binding of cAMP by release of catalytically active monomeric C subunit [3]. Reactivation of motility of demembranated sperm can be achieved either by cAMP or by active C subunit [4]. Although the underlying mechanisms are still unknown, sperm have been shown to contain a distinct adenylyl cyclase [5;6], PKA type I and II isozymes [7] and phosphodiesterases [8] as well as distinct PKA C isoforms [9-11] implying that sperm have the machinery to generate cAMP and mediate its effects. Furthermore, anchoring of PKA through A-Kinase Anchoring Proteins (AKAPs) to distinct intracellular sites in sperm is believed to be important for regulating sperm motility since disruption of the AKAP-PKA interaction results in motility arrest [12]. Thus, PKA may be essential for sperm function and consequently for fertility.

There is a substantial complexity of PKA isozymes due to heterogeneity in catalytic (Cα, Cβ, Cγ, PrKX [13]) and regulatory (RIα,RIβ,RIIα, RIIβ) subunits. The various R and C subunits are encoded by distinct genes localized on different chromosomes [14]. Several variants of the catalytic subunit in various species have previously been reported. These include human Cα-2[15], Aplysia Cα-N2[16], ovine Cα-s [11], bovine Cβ2[17], mouse Cβ1, Cβ2 and Cβ3[18], as well as a pseudogene for murine C, Cx [19]. In addition, the PKA subunit isoforms show cell-specific expression and differential regulation in several cells and tissues. They also reveal different subcellular localization, mainly due to binding to AKAPs [20;21].

### SUMMARY OF THE INVENTION

The present invention include the cloning of a novel human Cα isoform, identified as, the human Cα-s protein on the basis of its similarity to a recently characterized Cα isoform which was identified by purification and peptide sequencing of Cα from ovine sperm [11]. The human Cα-s protein was present in testis and ejaculated sperm and Cα-s localized to the midpiece of human sperm. The present invention includes in this respect a cDNA sequence encoding a Cα-s protein and comprises the specific nucleotide sequence shown in SEQ ID NO:1, (underlined) wherein the said protein is a new splice variant of Cα catalytic subunit of cAMP dependent protein kinase.

The present invention is further directed to a vector comprising the said cDNA sequence. The invention also includes a protein characterised by the Cα-s protein specific amino acid sequence shown in SEQ ID NO:2 (underlined). The invention includes further use of Cα-s protein and cDNA sequence in preparation of pharmaceuticals, medicaments to manipulate the motility in sperm or for use as cotraceptive, as well as methods and testsytems for screening molecules interacting with Cα-s. The present invention includes also a kit.

### BRIEF DESCRIPTION OF THE DRAWINGS

FIG. 1. A) Sequence alignment of human Cα isoforms. The cloned Cα-s cDNA sequence was translated into its corresponding amino acid sequence. The peptide sequences of Cα (top) and Cα-s (bottom) that correspond to exons 1 (bold) and the first 28 amino acids of exon 2 (boxed) from the Cα gene are shown. B) Sequence comparison of human (top) and ovine (bottom) Cα-s peptide sequences. Vertical lines denote homology.

FIG. 2. A) Tissue distribution of human Cα mRNAs. A human multiple tissue blot (Clontech Inc., 2 μg polyA⁺ RNA per lane) was probed with [³²P]-labeled Cα-s specific (top panel) or a Cα-1 specific (bottom panel) probe (see Materials and Methods). Lines indicate migration of RNA molecular weight markers (kb). B) Germ cell suspensions from human testis were fractionated by centrifugal elutriation, and total RNA was extracted and subjected to Northern blot analysis with a [³²P]-labeled Cα-s specific probe. Fractions were analyzed by flow cytometry for DNA content and separated into haploid cells [round spermatids (RS)] and tetraploid cells [pachytene spermatocytes (PS)]. Diploid cells were considered to be spermatogonia, secondary spermatocytes, or contamination by somatic cells and leucocytes. Lane 1: 57% RS, 7% PS, lane 2: 41% RS, lane 3: 16% RS, 7% PS, lane 4: 21% PS, 3% RS, lane 5: 51% PS, 4% RS. 20 ug of total RNA was loaded with exception of lane 2 (10 ug). The probe detected the Cα-s mRNA of approx 2.8 kb (top arrow) as compared to migration of 18S rRNA (bottom arrow).

FIG. 3. Human Cα-s immunoreactive protein in sperm. Nitrocellulose-immobilized proteins from human cell and tissue lysates were subjected to immunoblot analysis using either the αCα-s antiserum (A) in the absence (upper panel) or presence (lower panel) of 2 mM peptide antigen, or an anti-C antibody reactive to all C-subunit isoforms (B). Lane 1: recombinant murine C standard (500 ng), lane 2: human testis (20 μg protein), lane 3: human sperm lysate (10⁶ sperm), lane 4: human T-cells (20 μg), lane 5: human B-cells (20 μg), lane 6: human brain (20 μg), lane 7: HeLa cells (20 μg), lane 8: recombinant human Cγ (100 ng).

FIG. 4. A) Subcellular localization of human Cα-s in sperm cells. Human motile sperm were extracted with 1% Triton X-100, fixed with ethanol on glass slides and subjected to immunofluorescense analysis using the αCα-s antiserum (red staining) in the absence (left panel) or presence of 2 mM peptide antigen (middle panel). Cells were concomitantly DNA-stained with Hoechst (blue). B) Detergent extraction of human sperm tails. Sperm tails were extracted with 1% Triton X-100 (lanes 1 and 2) or 1% Triton X-100 and 20 μM cAMP (lanes 3 and 4), fractionated into soluble (S) and unsoluble (P) fractions and subjected to immunoblotting using the anti Cα-s antibody.

### DETAILED DESCRIPTION OF THE INVENTION

The abbreviations used are: PKA, cAMP-dependent protein kinase; AKAP, A-kinase anchoring protein; C, catalytic -and; R, regulatory subunit of PKA; PBS, phosphate buffered saline.

The present invention demonstrates the cloning and characterisation of a partial cDNA-sequence encoding Cα-s, an alternatively spliced PKA Cα subunit which contains a novel N-terminus encoded by an alternative exon 1 of the Cα gene. The inventors demonstrate that the Cα-s mRNA is expressed in human male germ cells and that the Cα-s protein is present in ejaculated sperm. Furthermore, the results demonstrate Cα-s localization to detergent soluble and detergent-resistant structures in sperm midpiece. The length of the unique N-terminal 7 amino acid sequence of both ovine and human Cα-s is derived from exon I in the respective species [37]. In addition, the murine Cx pseudogene [19] translates into a predicted N-terminal region (MPSSSND) with the same length that shares homology (4 identical residues out of 6) to both ovine and human Cα-s. Thus, it is possible that the murine Cx pseudogene originated by retroposition of a Cα-s mRNA. This implies the expression of Cα-s in three different species, which would mean that Cα-s has been conserved during evolution. The present invention relates to a cDNA sequence encoding a Cα-s protein and comprises the specific nucleotide sequence shown in SEQ ID NO: 1 (underlined), wherein the said protein is a new splice variant of Cα catalytic subunit of cAMP dependent protein kinase. The present invention is further directed to a vector comprising the said cDNA sequence. The invention also includes a protein characterised by the Cα-s protein specific amino acid sequence shown in SEQ ID NO:2 (underlined). The invention includes further use of Cα-s protein and cDNA sequence in preparation of pharmaceuticals, medicaments to manipulate the motility in sperm or for use as cotraceptive, as well as methods and testsytems for screening molecules interacting with Cα-s. The present invention includes also a kit.

The inventors have previously described another testis-specific C subunit, the Cγ isoform [9], which is present only in primates and is derived from an intronless retroposon [10]. Sequence alignment demonstrated that Cα-s is clearly distinct from the Cγ isoform, in agreement with a previous report on ovine Cα-s [11]. However, the expression pattern of Cα-s seen in human male germ cells resembles that of Cγ, and it is possible that these two isoforms utilize similar germ cell-specific promoter elements. Although the exact role of these proteins in human spermatogenesis and sperm function remains to be elucidated, the presence of two distinct C isoforms in the human testis and sperm suggest either a need for substantial redundancy in order to preserve function, or support a notion that different isoforms and alternatively spliced gene products serve distinct and specific sperm functions.

The solubility properties of human Cα-s are reflected by the observation that the presence of both Triton X-100 and cAMP was inadequate to fully extract this protein from sperm midpiece. This is in contrast to the previous report on ovine Cα-s [11] where ovine Cα-s could be extracted only by Triton X-100 when added in combination with cAMP. However, the authors did not account for the insoluble material after this extraction. Thus, it is possible that a pool of Cα-s is insoluble in the presence of cAMP also in ovine sperm. Nevertheless the inventors data support the notion that the Cα-s subunit has unusual solubility properties. An intriguing possibility is that in addition to its anchoring to an R subunit, the shortened Cα-s amino terminus (as compared to the “normal length” Cα), exposes hydrophobic regions that interacts with membraneous or cytoskeletal structures [32]. This would render the free monomeric Cα-s insoluble both in the absence and presence of cAMP. Such a hypothesis is supported by the fact that Cα-s lacks the N-terminal phosphorylation site at Ser10, present in other isoforms of the catalytic subunit [33]. Mutagenesis at Ser10 renders the catalytic subunit insoluble [34] possibly as a result of exposing hydrophobic regions. Independent anchoring of Cα-s may also provide an explanation to the fact that RII knock out mice are fertile [35]. The inventors have recently shown that the RIα and RIIα subunits of PKA are anchored by the A-kinase anchoring protein, AKAP220, to detergent-resistant structures in sperm midpiece whereas S-AKAP84 associate with membrane structures in the same region [36]. Thus, AKAP220/S-AKAP84, RIα/RIIα and Cα-s may constitute signalling complexes present in the midpiece region of human sperm specialised at regulating motility and/or other sperm functions.

In order to determined the functional importance of Cα-s in sperm motility one of the inventors generated knock out mice harbouring a genetic deletion in the gene encoding both variants of Cα, Cα1 and Cα-s (Skalhegg et al, 20000 submitted). Said mice develop morphological normal sperm with a normal appearance. However, the sperm have no motility capacity and PKA phosphotransferase activity. Due to the sperm motility defect, it is assumed that these mice are unfertile. The Cα null mutated mice have in addition 30% growth retardation due to low level of growth hormone. These mice are also sensitive to diseases and only 10% reach sexual maturity. Cα-s is an enzyme that is uniquely expressed in the midpiece of elongated mature sperm cells, whereby its function is to regulate sperm motility. Thus, altered levels, location and/or activity of Cα-s will according to our results, modulate the sperm cells ability to swim and fertilize the egg cell. This knowledge can be used to diagnose male infertility, treat cases of male infertility, and to develop male contraceptives.

1) Sperm cell motility and diagnostics: Sperm cell motility-dependent infertility may be caused by mal- or low enzymatic activity by Cα-s, the absence of Cα-s protein or dislocation of Cα-s.

1.1) Improving sperm cell motility: Present invention makes it possible to identify, characterize and produce pharmacological compositions after high through put screening that specifically will improve the enzymatic activity of Cα-s. These compositions should be developed such that they can be introduced directly (by mixing) to the sperm cells in order to improve motility in the context of in vitro fertilization or aided fertilization.

1.2) Kits for diagnosing non-motile sperm: Sperm cell motility-dependent infertility may be caused by absence of enzymatic active Cα-s. Present invention makes it possible to develop kits, which would diagnostically facilitate if the presence of Cα-s is the cause for non-motile sperm cells. Such kits should be developed with Cα-s specific antibodies, which have already been developed in the inventors laboratory.

2) Contraceptives based on Cα-s activity and location: Removing Cα-s activity and/or protein from the sperm midpiece renders the sperm cell non-motile as shown by generating genetic knock out mice lacking the Cα gene.

2.1) Contraceptives based on Cα-s activity: The present invention makes it possible to identify, characterize and produce pharmacological compositions after high through put screening that specifically and irreversibly will inhibit the enzymatic activity of Cα-s in newly synthesised sperm. These compositions should be developed such that they can be introduced orally to enter the blood system reaching the sperm cells.

2.2) Contraceptives based on Cα-s location: The present invention makes it possible to identify, characterize and produce pharmacological compositions after high through put screening that will specifically and irreversibly block Cα-s interaction with the sperm midpiece. These compositions should be developed such that they can be introduced orally to enter the blood system reaching the newly synthesised sperm cells.

The present invention makes it possible to develop a method for inspection and screening of patient spermatozoa for the presence and location of Cα-s protein comprising:

a) collecting and washing in buffer of ejaculated mature sperm or elongated spermatozoa directly isolated from the epididemys;

b) preparing of purified sperm cells for immunofluorescence by letting them settle onto poly L-lysine coated cover slips following detergent-dependent stripping of the sperm cells;

c) incubating with primary antibody (Ab), either irrelevant Ab or Cα-s specific Ab; Ab overshoot will be removed by washing buffer and sperm cells incubated with secondary anti-IgG Ab conjugated with a fluorescent;

d) inspection of sperm cells under fluorescent microscopy.

The present invention makes it further possible to develop a method for screening patient spermatozoa for the catalytic activity of the midpiece associated Cα-s comprising:

a) collecting and washing in buffer of ejaculated mature sperm or elongated spermatozoa directly isolated from the epididemys;

b) preparing of purified sperm cells for protein kinase assay by separating head and tail fraction by sonication, sentrifugation and lysis of sperm tail in detergent containing buffer;

c) monitoring Cα-s specific catalytic activity by established assay. Cα1 activity is used as an internal control to determine relative activity.

### Materials and Methods

Isolation of cDNA Clones by 5′-Rapid Amplification of cDNA Ends (RACE)-Human testis RACE-Ready cDNA was purchased from Clontech Laboratories Inc. Palo Alto Calif. (cat. no. 7414-1). A primary PCR with primer Cα-2137 (5′-GGACTTGGCCTCTCCTGTTCCCTTTTG-3′, nucleotides 2137 to 2163 in the human Cα cDNA sequence) or primer Cα-1363 (5′-TGTGGGGAAAGAGGAAGGGAAAAGT-3′, nucleotides 1363 to 1387 the human Cα cDNA sequence) and anchor specific primer (AP1, Clontech) was performed. A subsequent secondary PCR was performed using primer Cα-188 (5′-AAACTGATCCAAGTGGGCTGTGTTC-3′, nucleotides 188 to 212 in the human Cα cDNA sequence) or primer Cα-624 (5′-GCGAAACCGAAGTCTGTCACCTG-3′, nucleotides 624 to 646 in the human Cα cDNA sequence), and anchor specific primer (AP2, Clontech). The Clontech Advantage PCR kit (Clontech cat. no. K1905-1) was used in both PCR reactions with 25 cycles of denaturation at 94° C. for 30 s and with annealing and extension at 68° C. for 4 min. The 848- and 560-base pair PCR products were subcloned and sequenced.

Sequencing of cDNA Clones-Plasmids were sequenced on both strands by the dideoxy chain termination method (22) using cycle sequencing protocols (ThermoSequenase kit cat. no. US79760, Amersham Life Science Inc., Cleveland, Ohio 44128), [³³P]-labeled dideoxynucleotides (cat. no. AH9539, Amersham) and a combination of vector- and insert specific primers. Nucleotide and amino acid sequence data were analyzed using the GCG program package (program manual for the Wisconsin package, version 8, September 1994, Genetics Computer Group, Madison, Wis. 53711).

Fractionation of Germ Cells from Human Testis-Germ cells were isolated by combined treatment with trypsin and DNase [23], followed by separation using a centrifugal elutriation method essentially as described elsewhere [24]. Purity of the cell fractions was evaluated by analysis of DNA content by flow cytometry, and phase contrast microscopy.

Sperm fractionation—Mature motile sperm from human donors were purified using the swim-up procedure [25]. Removal of sperm heads was performed by sonicating sperm for 3 min. and subsequent sedimentation of heads at 1500×g for 5 min. For Triton X-100 extraction, sperm or sperm tails were extracted 30 min with 1% Triton X-100 in the presence or absence of 20 μM cAMP essentially as described elsewhere [11].

Oligo nucleotide probes-Specific probes were made using oligo 5′-AAGAAGGGCAGCGAGCAGGAGGCGTGAAAGAATTCTTAG-3′corresponding to positions 102 to 141 in the human Cα cDNA sequence (Cα-1) and oligo 5′-CTGAGAACAGGACTGAGTGATGGCTTCCAACTCCAGCGAT-3′, corresponding to positions 1 to 40 in the human Cα-s cDNA sequence (Cα-s), in an oligomerization protocol as described elsewhere [26].

Northern Analysis—Total RNA from human germ cell fractions was extracted by the guanidine isothiocyanate/CsCl method as previously described [27]. Northern analysis was performed using 10-20 μg RNA/lane. Ethidium bromide staining of the gel verified the loading in each lane. Filters with human germ cell fractions or multiple human tissues (Clontech, cat. no. 7759-1) were probed with [³²P]-labeled Cα-1 or Cα-s probes.

Antibodies—Anti-Cα-s antiserum was made by immunizing rabbits with hemocyanine-coupled synthetic peptides (Antigen EP 990209, NH₂-ASNSSDVKE-CONH₂; Eurogentec, Seraing, Belgium) corresponding to amino acids 1 to 9 of Cα-s and used at a dilution of 1/100 for immunofluorescence. Anti-Cα-s antiserum was enriched for IgG on protein A sepharose columns (Pharmacia, Stockholm, Sweden) and to subsequently affinity-purified on columns with peptide coupled to CNBr-activated Sepharose 4B (Pharmacia) and used at 2 μg/ml for immunoblotting. Cγ antibody (Santa Cruz Biotechnology, Inc., Santa Cruz Calif., USA, cat. no. sc-905) was used at 2 μg/ml to detect all isoforms of PKA C subunits.

Immunological procedures—Immunoblot analysis was performed as described elsewhere [28]. Immunoreactive proteins were detected by horseradish peroxidase-labeled protein A (Amersham) in the second layer and developed using ECL (Amersham cat. no. RPN2106). Immunofluorescence was performed on detergent extracted mature motile sperm from human donors (see above) fixed on poly-L lysine coated glass slides with 3% paraformaldehyde and permeabilized with 0.5% Triton X-100 for 15 min. Immunofluorescence detection was performed as described previously [29]. DNA was labeled with 0.1 μg/ml Hoechst 33342. Cells were examined on an Olympus AX70 epifluorescence microscope using a 100X objective. Photographs were taken with a Phototonic Science CCD camera and the OpenLab software (Improvision Co., Coventry, UK).

In order that this invention may be better understood, the following examples are set forth. These examples are for the purposes of illustration only, and are not to be construed as limiting the scope of the invention in any manner.

### EXAMPLES

### Example 1

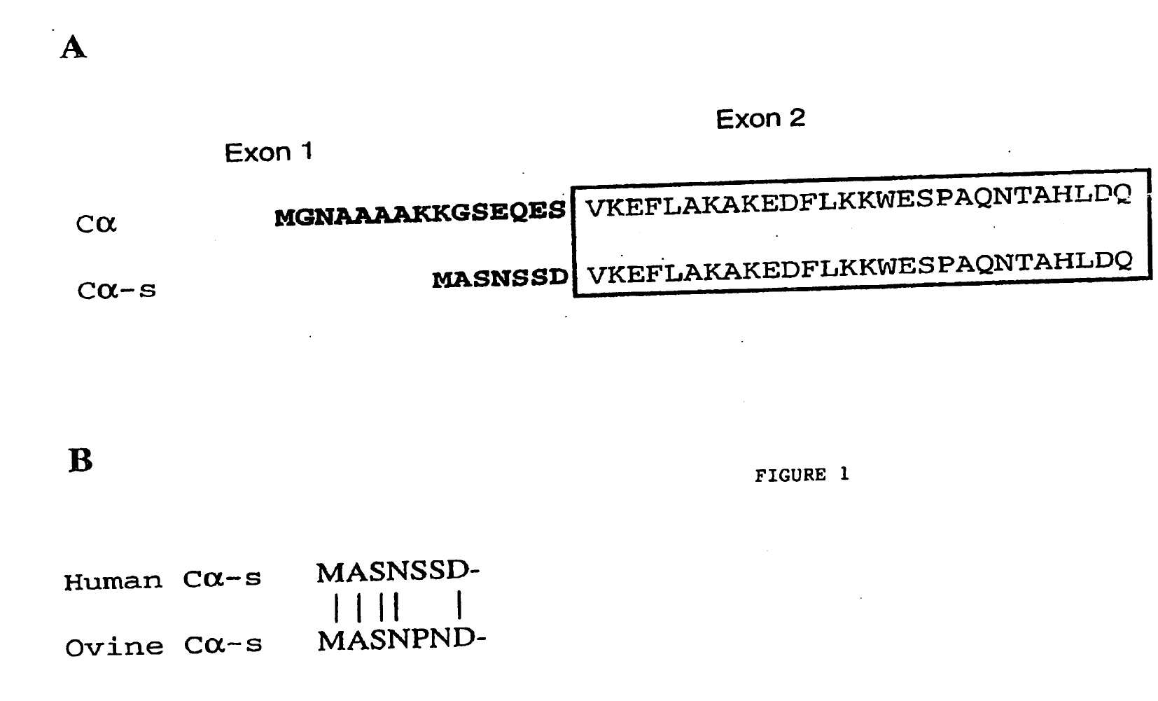

Identification of human Cα-s. In order to look for the possible presence of differentially spliced Cα transcripts, 5′-RACE was performed using a human testis cDNA library and primers situated downstream of exon 1. This yielded one clone of 560 bp where the 3′ region (nucleotides 40 to 560) shared identity to the previously characterized human Cα-cDNA [30] from nucleotide 126 to 646. Using the BLASTN program [basic local alignment and search tool [31]] similarity to the murine Cx pseudogene [19] was found from position 14 to 560. An ATG start codon was identified (positions 19 to 21), giving rise to 180 amino acids of reading frame that did not contain any stop. Whereas the 173 C-terminal amino acid residues of this partial reading frame was identical to positions 16 to 188 in the human Cα protein, the 7 amino acids (SEQ ID NO: 2, underlined) not present in the previously reported Cα sequence constituted a novel N-terminus. FIG. 1A shows the amino acid sequences corresponding to exon 2 in human Cα (boxed) and the N-terminal amino acids that differ between the two peptide sequences (bold). The N-terminal 7 amino acids were homologous (4 identical amino acids out of 6, in addition to the methionine) to a distinct Cα protein, Cα-s, purified and peptide sequenced from ovine sperm [11](FIG. 1B), demonstrating that the human Cα splice variant most probably is the Cα-s isoform.

### Example 2

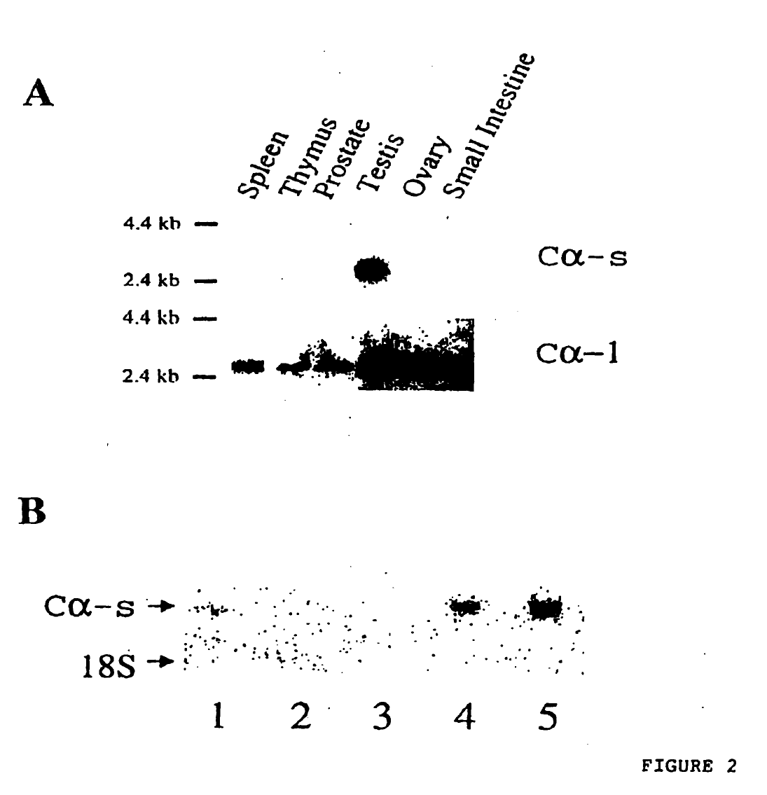

Tissue and Cell Specific Expression of Human Cα-s mRNA. To determine which human tissues expressed Cα and Cα-s mRNAs, specific probes for Cα (now designated Cα-1) and Cα-s were radioactively labeled and used to probe a Northern blot with human tissues. As shown in the top panel of FIG. 2A, Cα-s mRNA was expressed exclusively in testis. The Cα-1 probe detected expression in all tissues and cell types examined (FIG. 2A, lower panel). To further explore Cα-s mRNA expression in human testis, germ cells were examined for the expression of the Cα-s mRNA (FIG. 2B). Male human germ cells were isolated and fractionated as described in Materials and Methods. Cα-s mRNA was expressed in fractions 4 and 5, enriched in pachytene spermatocytes (PS) (lanes 4 and 5), but was not detected in fractions 1-3 enriched in round spermatids (RS) (lanes 1-3).

### Example 3

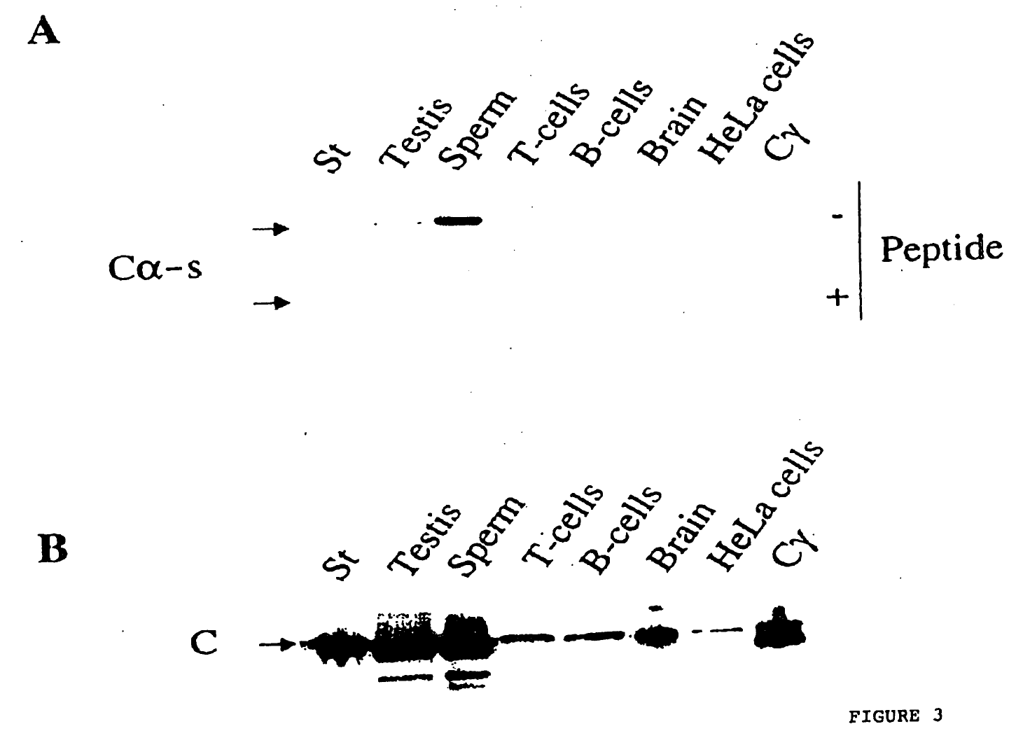

The Cα-s Protein Is Present in Human Testis and Sperm—An antiserum directed towards a peptide corresponding to amino acids 1 to 9 of Cα-s was affinity purified and used for immunoblotting analysis. Immunoblotting of human tissue and cell homogenates, revealed a 39-kDa immunoreactive band present in testis and sperm (FIG. 3A, top panel, lanes 2 and 3 respectively), but not in any other cells or tissues examined (FIG. 3A, top panel, lanes 4 to 7). As expected, heterologously expressed murine Cα and human Cα proteins did not react with the antiserum (FIG. 3A, lanes 1 and 8 respectively). The immunospecificity of the 39-kDa band in human testis and sperm was demonstrated by competition with the antigen (FIG. 3A, lower panel, lanes 2 and 3 respectively). The presence of other C isoforms in these tissues as well as immunoreactivity of the standards, was demonstrated by probing a parallel blot using an anti-C antibody that recognizes all known C isoforms (FIG. 3B).

### Example 4

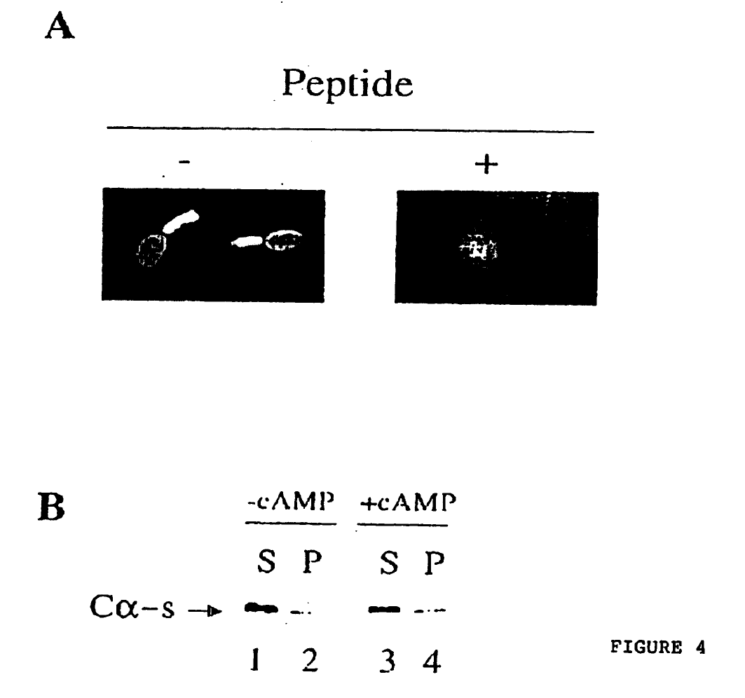

The Cα-s Protein Localizes to Detergent Resistant Structures in Sperm Midpiece—The subcellular localization of human Cα-s was examined in sperm by immunofluorescence using the Cα-s antiserum (FIG. 4A, red). In ejaculated sperm preparations extracted with Triton X-100 (FIG. 4A, left panel) or left untreated (results not shown), Cα-s labeling was restricted to the midpiece region. The staining was shown to be specific since preimmuneserum or an unrelated antibody did not produce any signal by immunofluorescence in sperm (results not shown), and the staining was competed with the corresponding peptide (FIG. 4A, right panel). To further examine the solubility of Cα-s in human sperm midpiece, we extracted sperm tails with detergent both in the absence and presence of cAMP. FIG. 4B shows immunoblotting of insoluble (P) and soluble (S) fractions from human sperm tails extracted with Triton X-100 (lanes 1 and 2) or Triton X-100 in combination with 20 μM cAMP (lanes 3 and 4), using the anti Cα-s antibody. In both cases approximately ⅔ could be solubilized from the particulate fraction. Addition of cAMP to the extraction buffer did not significantly alter the Cα-s signal in either fraction as compared to treatment with Triton X-100 alone, indicating that a pool of Cα-s was insoluble also in the presence of cAMP.

### References

[1] Tash J S, Means A R. Regulation of protein phosphorylation and motility of sperm by cyclic adenosine monophosphate and calcium. Biol. Reprod. 1982; 26: 745-763.

[2] Brokaw C J. A lithium-sensitive regulator of sperm flagellar oscillation is activated by cAMP-dependent phosphorylation. J. Cell Biol. 1987; 105: 1789-1798.

[3] Beebe S J, Corbin J D. Cyclic nucleotide-dependent protein kinases. The Enzymes 1986; 17: 43-111.

[4] San Agustin J T, Witman G B. Role of cAMP in the reactivation of demembranated ram spermatozoa. Cell Motil. Cytoskeleton 1994; 27: 206-218.

[5] Gordeladze J O, Hansson V. Purification and kinetic properties of the soluble Mn2+-dependent adenylyl cyclase of the rat testis. Mol. Cell Endocrinol. 1981; 23: 125-136.

[6] Buck J, Sinclair M L, Schapal L, Cann M J, Levin L R Cytosolic adenylyl cyclase defines a unique signaling molecule in mammals. Proc. Natl. Acad. Sci. U.S.A 1999; 96: 79-84.

[7] Landmark B F, Oyen O, Skalhegg B S, Fauske B, Jahnsen T. Hansson V. Cellular location and age-dependent changes of the regulatory subunits of cAMP-dependant protein kinase in rat testis. J. Reprod. Fertil. 1993; 99: 323-334.

[8] Salanova M, Chun S Y, Iona S, Puri C, Stefanini M, Conti M. Type 4 cyclic adenosine monophosphate-specific phosphodiesterases are expressed in discrete subcellular compartments during rat spermiogenesis. Endo 1999; 140: 2297-2306.

[9] Beebe S J, Øyen O, Sandberg M, Frøysa A, Hansson V, Jahnsen T. Molecular cloning of a tissue-specific protein kinase (C gamma) from human testis—representing a third isoform for the catalytic subunit of cAMP-dependent protein kinase. Mol. Endocrinol. 1990; 4: 465-75.

[10] Reinton N. Haugen T B, Orstavik S, Skalhegg BS, Hansson V, Jahnsen T, Tasken K. The gene encoding the C gamma catalytic subunit of cAMP-dependent protein kinase is a transcribed retroposon. Genomics 1998; 49: 290-297.

[11] San Agustin J T, Leszyk J D, Nuwaysir L M, Witman G B. The catalytic subunit of the cAMP-dependent protein kinase of ovine sperm flagella has a unique amino-terminal sequence. J. Biol. Chem. 1998; 273: 24874-24883.

[12] Vijayaraghavan S, Goueli S A, Davey M P, Carr D W. Protein kinase A-anchoring inhibitor peptides arrest mammalian sperm motility. J. Biol. Chem. 1997; 272: 4747-4752.

[13] Zimmermann B, Chiorini J A, Ma Y, Kotin R M, Herberg F W. PrKX is a novel catalytic subunit of the cAMP-dependent protein kinase regulated by the regulatory subunit type I. J. Biol. Chem. 1999; 274: 5370-5378.

[14] Tasken K, Naylor S L, Solberg R, Jahnsen T. Mapping of the gene encoding the regulatory subunit RII alpha of cAMP dependent protein kinase (locus PRKAR2A) to human chromosome region 3p21.3-p21.2. Genomics 1998; 50: 378-381.

[15] Thomis D C, Floyd-Smith G. Samuel C E. Mechanism of interferon action. cDNA structure and regulation of a novel splice-site variant of the catalytic subunit of human protein kinase A from interferon-treated human cells. J. Biol. Chem. 1992, 267: 10723-10728.

[16] Beushausen S, Lee E, Walker B, Bayley H. Catalytic subunits of Aplysia neuronal cAMP-dependent protein kinase with two different N termini. Proc. Natl. Acad. Sci. U.S.A 1992; 89: 1641-1645.

[17] Wiemann S, Kinzel V, Pyerin W. Isoform Cβ2, an unusual form of the bovine catalytic subunit of cAMP-dependent protein kinase. J. Biol. Chem. 1991; 266: 5140-5146.

[18] Guthrie C R, Skalhegg B S, McKnight G S. Two novel brain-specific splice variants of the murine Cbeta gene of cAMP-dependent protein kinase. J. Biol. Chem. 1997, 272: 29560-29565.

[19] Cummings D E, Edelhoff S, Disteche C M, McKnight G S. Cloning of a mouse protein kinase A catalytic subunit pseudogene and chromosomal mapping of C subunit isoforms. Mamm. Genome 1994; 5: 701-706.

[20] Scott J D. Cyclic nucleotide-dependent protein kinases. Pharmacol. Ther. 1991; 50: 123-145.

[21] Rubin C S. A kinase anchor proteins and the intracellular targeting of signals carried by cyclic AMP. Biochim. Biophys. Acta 1994; 1224: 467-479.

[22] Sanger F, Nicklen S, Coulson A R. DNA sequencing with chain-terminating inhibitors. Proc. Natl. Acad. Sci. U.S.A. 1977; 74: 5463-5467.

[23] Grootegoed J A, Peters M J, Mulder E, Rommerts F F, van der Molen H J. Absence of a nuclear androgen receptor in isolated germinal cells of rat testis. Mol. Cell Endocrinol. 1977; 9: 159-167.

[24] Blanchard Y, Lavault M T, Quernee D, Le Lannou D, Lobel B, Lescoat D. Preparation of spermatogenic cell populations at specific stages of differentiation in the human. Mol. Reprod. Dev. 1991; 30: 275-282.

[25] Wikland M, Wik O, Steen Y, Qvist K, Soderlund B, Janson P O. A self-migration method for preparation of sperm for in-vitro fertilization. Hum. Reprod. 1987; 2: 191-195.

[26] Paquette J, Sapienza C. A reliable method for the use of oligonucleotides as probes in blot-hybridization experiments. Mamm. Genome 1992; 3: 1-4.

[27] Knutsen H K, Reinton N, Tasken K A, Hansson V, Eskild W. Regulation of protein kinase A subunits by cyclic adenosine 3′, 5′-monophosphate in a mouse Sertoli cell line (MSC-1): induction of RII beta messenger ribonucleic acid is independent of continuous protein synthesis. Biol. Reprod. 1996; 55: 5-10.

[28] Witczak O, Skalhegg B S, Keryer G, Bornens M, Tasken K, Jahnsen T, Orstavik S. Cloning and characterization of a cDNA encoding an A-kinase anchoring protein located in the centrosome, AKAP450. EMBO J. 1999; 18: 1858-1868.

[29] Collas P, Courvalin J C, Poccia D. Targeting of membranes to sea urchin sperm chromatin is mediated by a lamin B receptor-like integral membrane protein. J. Cell Biol. 1996; 135: 1715-1725.

[30] Maldonado A, Hanks S K. A cDNA clone encoding human cAMP-dependent protein kinase catalytic subunit Ca. Nucl. Acids Res. 1988; 16: 8189-8190.

[31] Altschul S F, Gish W, Miller W, Myers E W, Lipman D J. Basic local alignment search tool. J. Mol. Biol. 1990; 215: 403-410.

[32] Herberg F W, Zimmermann B, McGlone M, Taylor S S. Lnportance of the A-helix of the catalytic subunit of cAMP-dependent protein kinase for stability and for orienting subdomains at the cleft interface. Protein Sci. 1997; 6; 569-579.

[33] Yonemoto W. Garrod S K Bell S M, Taylor S S. Identification of phosphorylation sites in the recombinant catalytic subunit of cAMP-dependent protein kinase. J. Biol. Chem. 1993; 268: 18626-18632.

[34] Yonemoto W, McGlone M L, Grant B. Taylor S S. Autophosphorylation of the catalytic subunit of cAMP-dependent protein kinase in Escherichia coli. Protein Eng 1997; 10: 915-925.

[35] Burton K A, Treash-Osio B, Muller C H, Dunphy E L, McKnight G S. Deletion of Type IIalpha Regulatory Subunit Delocalizes Protein Kinase A in Mouse Sperm without Affecting Motility or Fertilization. J. Biol. Chem. 1999; 274: 24131-24136.

[36] Reinton, N., P. Collas, T. Haugen, B. S. Sklhegg, V. Hansson, T. Jahnsen and., Taskén, K. A human A-kinase anchoring protein, hAKAP220, localizes to male germ cell centrosomes and sperm midpiece. Dev Biol 2000; 223, 194-204.

[37] Desseyn, J. L., Burton, K. A., & McKnight, G. S. (2000) Expression of a nonmyristylated variant of the catalytic subunit of protein kinase A during male germ-cell development. Proc. Natl Acad Sci. U.S.A, 97, 6433-6438.

1. A cDNA sequence encoding a Cα-s protein comprising the nucleotide sequence SEQ ID NO 1, wherein said protein is a new splice variant of a Cα catalytic subunit of cAMP dependent protein kinases.

2. A vector comprising the cDNA sequence according to claim 1

3. The specific amino acid sequence of SEQ ID NO: 2.

4. A protein encoded by the nucleotide sequence of claim 1.

5. A protein encoded by the cDNA sequence of claim 1 comprising the specific Cα-s protein amino acid sequence of SEQ ID NO: 2.

6. A kit comprising antibodies against the Cα-s protein of claims 1-5.

7. The kit according to claim 6 for diagnosing non-motile sperm.

8. The use of the Cα-s protein of claims 1-5, for the preparation of a pharmaceutical.

9. The use of the Cα-s protein of claims 1-5, for the preparation of a medicament for manipulating the motility in sperm.

10. The use of Cα-s protein of claims 1-5, for the preparation of a medicament for use as contraceptive.

11. The use of the Cα-s protein of claims 1-5, for the preparation of a medicament for the treatment of male infertility.

12. The use of a DNA sequence which is complementary to the Cα-s DNA sequence according to claim 1 for the preparation of an anti sense drug.

13. A method for inspection and screening of patient spermatozoa for the presence and location of Cα-s protein of claims 1-5, comprising:
a) collecting and washing in buffer of ejaculated mature sperm or elongated spermatozoa directly isolated from the epididemys;
b) preparing purified sperm cells for immunofluorescence by letting them settle onto poly L-lysine coated cover slips following detergent-dependent stripping of the sperm cells;
c) incubating with primary antibody (Ab), either irrelevant Ab or Cα-s specific Ab, Ab overshoot will be removed by washing buffer and sperm cells incubated with secondary anti-IgG Ab conjugated with a fluorescent;
d) inspection of sperm cells under fluorescent microscopy.

14. Method of screening patient spermatozoa for the catalytic activity of the midpiece associated Cα-s of claims 1-5, comprising:
a) collecting and washing in buffer of ejaculated mature sperm or elongated spermatozoa directly isolated from the epididemys;
b) preparing of purified sperm cells for protein kinase assay by separating head and tail fraction by sonication, sentrifugation and lysis of sperm tail in detergent containing buffer;
c) monitoring Cα-s specific catalytic activity by established assay, Cα1 activity is used as an internal control to determine relative activity.

15. A product produced by the method according to claim 12 or 13.

16. A test system using inhibitory- or activating molecules of the Cα-s protein of claims 1-5, for screening of male patients suffering sperm-motility-dependent infertility for defects in Cα-s activity and/or location using assay that will measure PKA catalytic activity.

17. The product from the screening to claim 16.
